(19) United States
(12) Patent Application Publication
(10) Pub. No.: US 2004/0103897 A1
(43) Pub. Date: Jun. 3, 2004

(54) Drug delivery system and method

(76) Inventors: Randall S. Hickle, Lubbock, TX (US); Michael Gustafson, Cincinnati, OH (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/660,201

(22) Filed: Sep. 11, 2003

Related U.S. Application Data
(60) Provisional application No. 60/411,077, filed on Sep. 16, 2002.

(51) Int. Cl. A61M 16/00

(52) U.S. Cl. 128/204.23

(57) ABSTRACT

A drug delivery system is provided for use in a sedation and analgesia system. The drug delivery system can include a first bolus interface, a second bolus interface wherein the second bolus interface appears after the first bolus interface, a third bolus interface wherein the third bolus interface can appear after the second bolus interface, and a drug delivery display. The first bolus interface asks a user if he or she wants to give the patient a bolus from the drug delivery. If the user answers yes, then the second bolus interface asks the user to confirm the bolus. The third bolus interface can appear if the bolus is outside a predetermined range flagging the user that the bolus is outside a specific range asking for confirmation to continue. The drug delivery display can show the drug delivery or any other patient statistics, but is not limited to, graphically or numerically

### CROSS REFERENCE TO RELATED APPLICATIONS

This application claims the benefit and priority from U.S. provisional application Serial No. 60/411,077, filed on Sep. 16, 2002, which is incorporated by reference herein in its entirety. The present application cross references and incorporates by reference copending U.S. Ser. No. 09/324,759, filed Jun. 3, 1999, U.S. Serial No. 60/330,853, filed Nov. 1, 2001.

### FIELD OF THE INVENTION

The present invention relates, in general, to drug delivery and, more particularly, to bolus drug delivery in conjunction with integrated patient monitoring and drug delivery systems.

### BACKGROUND OF THE INVENTION

Sedation and analgesia systems were developed to provide patients undergoing painful, uncomfortable or otherwise frightening (anxiety inspiring) medical or surgical procedures with a means for receiving sedative, analgesic, and/or amnestic drugs safely in a way that reduces the risk of overmedication with or without the presence of a licensed anesthesiologist. Due to the reduced number of potential failure modes commonly associated with anesthesia machines, sedation and analgesia systems have become safer for use in hospital and ambulatory environments and may be operated by individuals other than trained anesthesiologists such as, for example, C.R.N.A.'s, trained physicians, or other licensed operators. Sedation and analgesia systems have gone far to meet the anesthesia needs of office based practitioners who are unable to afford or schedule anesthesiologists for every procedure where the effects of sedation and analgesia would be beneficial. The advent of a sedation and analgesia system devoted to these purposes provides these individuals with a drug delivery system integrated into a

patient monitoring system that decreases the manual decision making required by anesthesia machines, yet gives the physician ultimate decision making responsibility following a “physician knows best” philosophy. The reduction of many manual activities associated with anesthesia machines allows for a sedation and analgesia system to be operated without an anesthesiologist in ambulatory settings providing the patient with a cost-effective and readily available means of sedation.

Since the inception of sedation and anesthesia, a number of methods of drug delivery have been developed in attempts to ensure patient comfort during painful, uncomfortable, or anxiety inspiring medical procedures. One such method, commonly used in fields such as gastroenterology, involves the serial delivery of bolus drug doses to a patient. In such cases, a patient is given an initial bolus drug infusion, where the amount of drug delivered is based on an estimation of the amount of drug needed to properly sedate and/or provide analgesia for a patient given certain physical parameters such as, for example, height and weight. Further bolus drug infusions are often given periodically throughout the procedure as the clinician recognizes signs that the patient is anxious or experiencing pain. Providing bolus drug infusions in such a manner generally requires the clinician to overshoot the target drug level in order to provide the patient with appropriate levels of sedation and/or analgesia. Overshooting the target drug level may result in a drug overdose, where a patient may suffer adverse consequences such as, for example, airway obstruction and hemoglobin desaturation. Under-medication may also occur in the bolus method, where a clinician may not administer a new bolus infusion until a patient is experiencing significant anxiety and/or pain.

A number of devices and methods for drug delivery have been developed in attempts to decrease the incidence and negative effects of patient overdose. Microcomputers and programmable controllers have been implemented into existing drug delivery systems in attempts to maintain a target controlled infusion (TCI) of drugs such as sedatives, analgesics, and amnestics. Unlike systems relying solely upon intermittent bolus administration, TCI systems attempt to maintain a constant level of drug effect by delivering drugs at a controlled rate. The TCI rate is often estimated by factoring in a patient's physical parameters such as, for example, height, weight, age, and gender, as well as the prior history of drug administration. Though TCI systems have had some success in maintaining constant patient drug states, a substantial amount of time is often required for the system to bring the patient up to the desirable level of sedation or general anesthesia. Unnecessary time spent awaiting a patient to become sedated is often undesirable, because on some occasions, physicians must quickly manage the pain and anxiety associated with painful procedures in order to minimize patient risks. In addition, unnecessary time spent awaiting a patient to become sedated is inefficient utilization of medical facilities and clinician's time.

In response to the need to sedate or anesthetize patients quickly, TCI systems usually have integrated initial bolus drug delivery or an initial increased infusion rate. TCI systems incorporating an initial bolus drug delivery generally comprise the delivery of a bolus infusion, where the drug amount needed to quickly reach the target threshold infusion rate is estimated based on a patient's physical parameters such as, for example, height and weight. Following the initial bolus delivery, a calculated infusion of a desirable drug is then administered to the patient to achieve the targeted drug level. A second method employed to reduce the time needed for a patient to reach a target sedation level generally comprises providing a system with two infusion rates, where the initial infusion rate delivers substantially more drug in relation to time than the secondary infusion rate. The initial infusion rate is delivered at a rate estimated by the clinician to quickly bring the patient to the target threshold, at which point the clinician will then switch to the secondary infusion rate to maintain the desired level of sedation or anesthesia.

Though such systems have had some success in quickly sedating and anesthetizing a patient without significant overmedication or under-medication, there are a number of procedures where a consistent drug infusion rate is undesirable. For example, a number of especially painful procedures such as cardiac cardioversions are characterized by a very brief but very painful stimulus. Thus, procedures of this type require a brief increase in drug level to minimize patient anxiety, pain, and unpleasant memories of the event. Though current TCI systems have had some success in sedating or anesthetizing patients during procedures where the patient experiences modes and variance in anxiety and/or pain, such TCI systems may not optimally manage the pain and/or anxiety needs of a patient undergoing a procedure that has brief, yet highly painful or anxiety inspiring periods, where a calculated and precise increased level of drug administration, followed by an immediate decrease of drug levels, may be desirable.

The DIPRIFUSOR, a trademark of Astra-Zeneca, is a target controlled infusion system, where drug delivery rate is based on a pharmacokinetic model designed to achieve a desired drug concentration in the blood of a patient. Once the targeted blood concentration is reached, the concentration of drug in the effect site (e.g., the brain) will slowly begin to reach equilibrium with the drug concentration found in the blood. The DIPRIFUSOR further includes an initial bolus drug delivery capability, where a bolus infusion may be made at the beginning of a procedure by the user to quickly reach a targeted blood concentration of administered drug. Though TCI systems such as the DIPRIFUSOR have had some success in meeting the sedation and analgesia needs of patients and clinicians, the time required for a patient to reach a desirable effect site concentration in, for example, the brain, is often undesirably long. The delay in reaching a desirable effect site concentration often found in such existing systems is generally due to the substantial time required for the effect site to reach equilibrium with the drug concentration in the blood, where the drug concentration in the blood is maintained at a level equal to the targeted effect site concentration. For example, if a desirable effect site concentration in the brain of a drug, such as the sedative propofol, for a gastroenterological procedure is 4.0 ug/cc, many existing systems infuse drugs at a rate that increases the concentration of administered drug in the blood to 4.0 ug/cc. In such cases, it will take several minutes for the effect site concentration to reach equilibrium with the drug concentration in the blood. The need has therefore arisen for a target controlled infusion model that quickly raises the effect site concentration of a patient's brain to a desirable level.

### BRIEF SUMMARY OF THE INVENTION

The present invention provides a drug delivery system that incorporates the benefits of an integrated patient monitoring system and that quickly brings a patient to the desired level of sedation or anesthesia while reducing the risk of overmedication or under-medication, while giving the clinician the capability to safely and efficiently deliver a precise and calculated bolus drug dosage at any point during the procedure. The present invention also provides a drug delivery system integrated with a patient monitoring system that establishes target infusion levels as a measure of the effect site concentration of critical patient areas such as, for example, the brain. The present invention further provides a system for safely delivering an increased drug dosage at any point during a medical procedure, where the increased drug dosage is stepped up in terms of the estimated increase in effect site concentration, rather than a volumetric dosage or a blood level concentration target. The present invention even further provides an easily accessible means of delivering an increased drug dosage at any point during a medical procedure, where existing systems generally allow a bolus drug infusion only at the beginning of a medical procedure.

### BRIEF DESCRIPTION OF THE FIGURES

FIG. 1 illustrates a block diagram of one embodiment of a sedation and analgesia systems having a user interface in accordance with the present invention.

FIG. 2 illustrates one embodiment of a drug delivery prompt in accordance with the present invention.

FIG. 3 illustrates one embodiment of a drug delivery prompt in accordance with the present invention.

FIG. 4 illustrates a further embodiment of a drug delivery prompt in accordance with the present invention.

FIG. 5 illustrates one embodiment of a drug delivery display in accordance with the present invention.

FIG. 6 illustrates one embodiment of a method for delivering a bolus drug infusion in accordance with the present invention.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

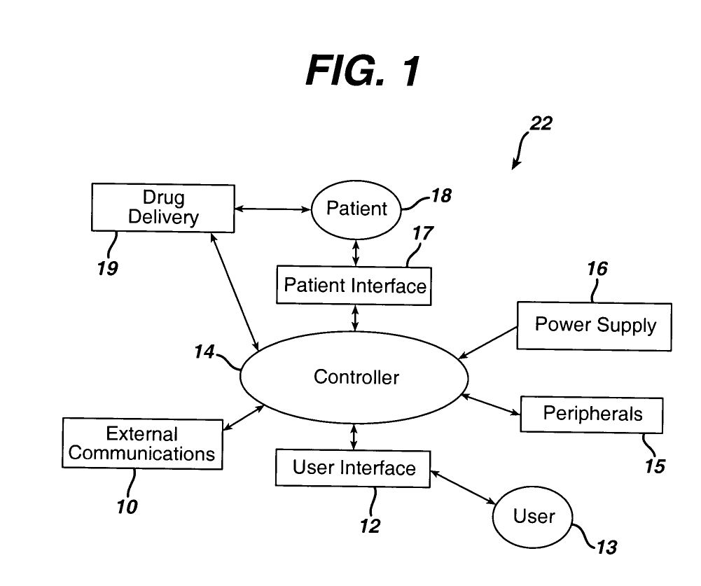

FIG. 1 illustrates a block diagram depicting one embodiment of the present invention comprising sedation and analgesia system 22 having user interface 12, software controlled controller 14, peripherals 15, power supply 16, external communications 10, patient interface 17, and drug delivery 19, where sedation and analgesia system 22 is operated by user 13 in order to provide sedation and/or analgesia to patient 18. An example of sedation and analgesia system 22 is disclosed and enabled by U.S. patent application Ser. No. 09/324,759, filed Jun. 3, 1999 and incorporated herein by reference in its entirety. Embodiments of user interface 12 are disclosed and enabled by U.S. Patent Application Serial No. 60/330,853, filed Nov. 1, 2001 and incorporated herein by reference in its entirety.

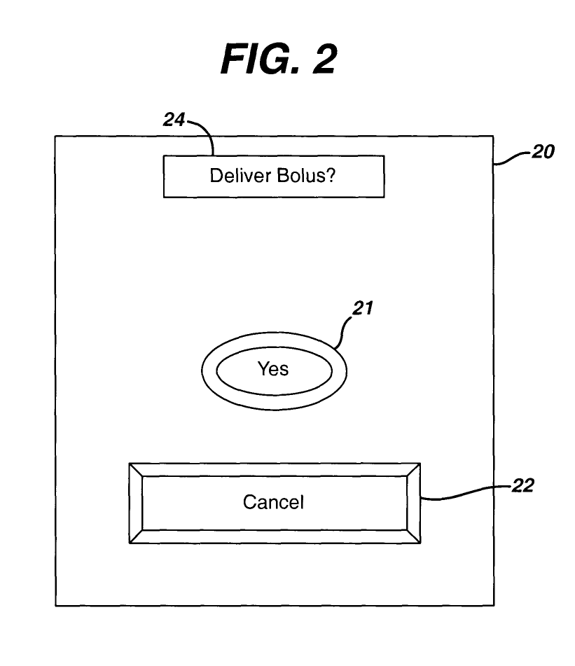

FIG. 2 illustrates one embodiment of first bolus delivery interface 20, where first bolus delivery interface 20 may be incorporated into user interface 12. First bolus delivery interface 20 may be a prompt found on a touch screen, a soft button interface, a hard button interface, or other suitable interface means. First bolus delivery interface 20 may further be part of a touch screen interface, where first bolus delivery interface is always present. A further embodiment of first bolus delivery interface comprises providing a hard button incorporated into user interface 12, where depression of the hard button prompts first bolus delivery interface 20. The present invention further comprises a plurality means of prompting first bolus delivery interface 20 such as, for example, by providing a small bolus icon on a touch screen interface, where touching the bolus icon prompts first bolus delivery interface 20.

In one embodiment of the present invention, first bolus delivery interface 20 comprises first text box 24, where first text box 24 queries user 13 whether they wish to deliver a bolus drug delivery. First text box 24 may contain any suitable text and/or symbol to query user 13. For example, an icon illustrating a bolus dosage may be presented to user 13, where user 13 may cancel or confirm the visual query. The present invention further comprises providing sedation and analgesia system 22 with multiple linguistic capabilities, where sedation and analgesia system 22 is capable of displaying text in a number of languages. The present invention further comprises incorporating first text box 24 into first key 21 and/or second key 22, where first key 21 may command user 13 to depress or otherwise signal first key 21 if they wish to deliver a bolus drug infusion and second key 22 may command user 13 to depress or otherwise signal second key 22 if they do not wish to deliver a bolus drug infusion.

First key 21 and/or second key 22 of first bolus delivery interface 20 may be touch screen buttons that are part of a touch screen display, buttons that are responsive to audio commands, soft buttons, hard buttons, or any other suitable means of inputting a command into sedation and analgesia system 22. In one embodiment of the present invention, first key 21 comprises signaling an affirmative response from user 13 to the query presented in first text box 24 and second key 22 comprises signaling a negative response from user 13 to the query presented in first text box 24. First key 21 and/or second key 22 may have textual indicators of their function such as, for example, “Yes” written on first key 21, or iconic indicators of their function such as, for example, an “X” written on second key 22 indicating a negative response. First text box 24, first key 21, and second 22 may be positioned at any suitable location on first bolus delivery interface 20 and/or user interface 12. In one embodiment of the present invention, sedation and analgesia system 22 may be operated at drug levels providing patient 18 with conscious sedation by any suitable trained clinician such as, for example, a suitable trained gastroenterologist. In a further embodiment of the present invention, sedation and analgesia system 22 may be operated at drug levels providing patient 18 with anesthesia by any clinician suitably trained in anesthetics such as, for example, certified registered nurse anesthetists (CRNAs) or anesthesiologists.

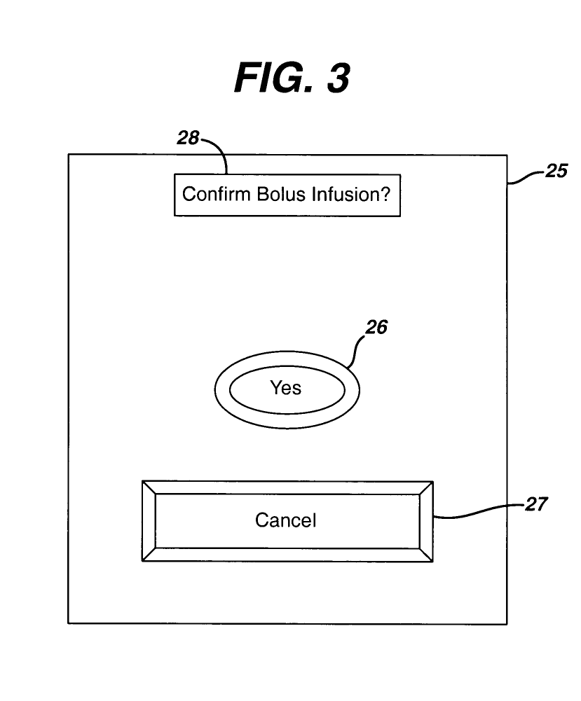

FIG. 3 illustrates one embodiment of second bolus delivery interface 25, where second bolus delivery interface 25 may be incorporated into user interface 12. Second bolus delivery interface 25 may be a prompt found on a touch screen, a soft button interface, a hard button interface, or other suitable interface means. Second bolus delivery interface 25 may further be part of a touch screen interface, where second bolus delivery interface 25 is prompted following an affirmative response to a bolus drug delivery associated with first bolus delivery interface 20.

In one embodiment of the present invention second bolus delivery interface 25 comprises second text box 28, where second text box 28 queries user 13 whether they wish to confirm a bolus drug infusion. Second text box 28 may contain any suitable text and/or symbol to query user 13. For example, an icon illustrating a confirmed bolus dosage may be presented to user 13, where user 13 may cancel or confirm the visual query. The present invention further comprises providing sedation and analgesia system 22 with multiple linguistic capabilities, where sedation and analgesia system 22 is capable of displaying text in a number of languages. The present invention further comprises incorporating second text box 28 into first key 26 and/or second key 27, where first key 26 may command user 13 to depress or otherwise signal first key 26 if they wish to confirm a bolus drug infusion and second key 27 may command user 13 to depress or otherwise signal second key 27 if they do not wish to confirm a bolus drug infusion.

First key 26 and/or second key 27 of second bolus delivery interface 25 may be touch screen buttons that are part of a touch screen display, buttons that are responsive to audio commands, soft buttons, hard buttons, or any other suitable means of inputting a command into sedation and analgesia system 22. In one embodiment of the present invention first key 26 comprises signaling an affirmative response from user 13 to the query presented in second text box 28 and second key 27 comprises signaling a negative response from user 13 to the query presented in second text box 28. First key 26 and/or second key 27 may have textual indicators of their function such as, for example, “Yes” written on first key 26, or iconic indicators of their function such as, for example, an “X” written on second key 27 indicating a negative response. Second text box 28, first key 26, and second key 27 may be positioned at any suitable location on second bolus delivery interface 25 and/or user interface 12. In one embodiment of the present invention, a bolus drug infusion is not given to patient 18 until user 13 confirms their initial drug delivery request. Providing a confirmation prompt may help to ensure that clinicians do not inadvertently signal a bolus drug infusion. The present invention further comprises displaying second bolus delivery interface 25 in a different location than first bolus delivery interface 20, where a clinician is required to look to a different part of user interface 12 to confirm the bolus drug infusion. Providing bolus delivery interfaces in different locations may help to prevent inadvertent confirmation of a bolus infusion due to rapid tapping of the interface buttons.

In one embodiment of the present invention, bolus drug infusions are delivered at a suitable rate to quickly increase the drug effect site concentration of patient 18. For example, initiating a single bolus drug infusion may cause drug delivery 19 to deliver enough drug to raise the effect site concentration of patient 18 by 0.5 ug/cc. If user 13 wishes to deliver a bolus infusion greater than 0.5 ug/cc they may be required to reprompt the bolus drug delivery interfaces and confirm a second bolus infusion that will increase the drug effect site concentration by another 0.5 ug/cc. By providing a bolus infusion system that targets sequential increases in the effect site concentration of patient 18, the present invention may decrease the chances of user 13 inadvertently inputting and confirming a potentially dangerous drug dosage. Programming associated with the amount of drug needed to reach a target effect site concentration may be integrated with controller 14, where controller 14 may rely on a pharmacokinetic model of drug infusion and patient parameters to correctly administer proper drug dosages.

Sedation and analgesia system 22 comprises pre-programmed safe drug levels given the physical parameters of a patient such as, for example, height, weight, sex, and age. At times during a medical procedure it may be beneficial to go outside the pre-programmed safe drug levels, however it may be beneficial to provide a prompt alerting user 13 that they are proceeding with drug infusion levels outside the pre-programmed safe range.

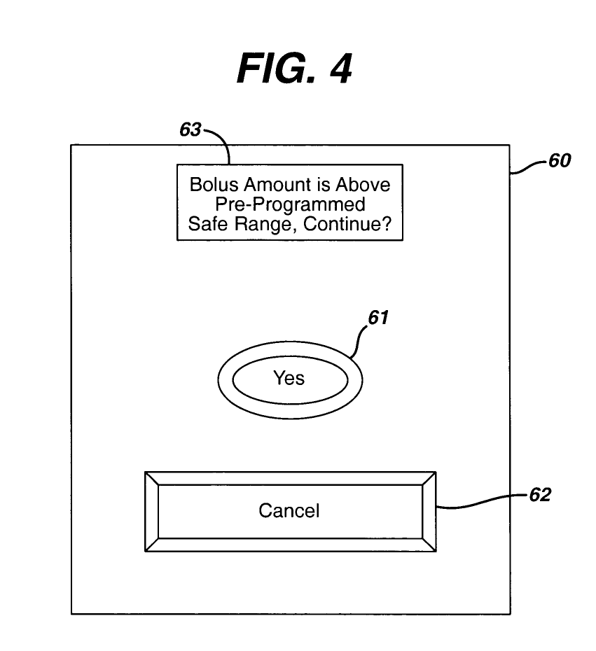

FIG. 4 illustrates one embodiment of third bolus delivery interface 60, where third bolus delivery interface 60 may be incorporated into user interface 12. Third bolus delivery interface 60 may be a prompt found on a touch screen, a soft button interface, a hard button interface, or other suitable interface means. In one embodiment of the present invention, third bolus delivery interface 60 is automatically prompted by sedation and analgesia system 22 following a confirmation of a bolus drug delivery outside the pre-programmed safe drug level range given based on the physical parameter of patient 18.

In one embodiment of the present invention third bolus delivery interface 60 comprises third text box 63, where third text box 63 queries user 13 whether they wish to confirm a bolus drug delivery outside of the pre-programmed safe drug level. First text box 33 may contain any suitable text and/or symbol to query user 13. For example, an icon illustrating that the bolus dosage is outside the pre-programmed range may be presented to user 13, where user 13 may cancel or confirm the visual query. The present invention further comprises providing sedation and analgesia system 22 with multiple linguistic capabilities, where sedation and analgesia system 22 is capable of displaying text in a number of languages. The present invention further comprises incorporating third text box 63 into first key 61 and/or second key 62, where first key 61 may command user 13 to depress or otherwise signal first key 61 if they wish to confirm a bolus drug infusion outside the pre-programmed level and second key 62 may command user 13 to depress or otherwise signal second key 62 if they do not wish to confirm a bolus drug infusion outside the pre-programmed level. Third text box 63 of third bolus delivery interface 60 further comprises text or icons indicating the maximum suggested drug level given a patient's physical parameters such as, for example, “The bolus drug infusion level is outside the pre-programmed safe level of drug suggested for patient's over the age of 70. Do you wish to confirm the bolus drug infusion?” If user 13 confirms the bolus drug infusion, the infusion will be given.

In a further embodiment of the present invention, third bolus delivery interface 60 comprises third text box 63, where third text box 63 may indicate to user that the bolus drug infusion request is outside a pre-programmed range, and that the bolus infusion will not be administered. For example, sedation and analgesia system 22 may be pre-programmed not to exceed a target site concentration drug level of 20 ug/cc, where user 13 is attempting to deliver a bolus drug infusion while patient 18 is currently at the 20 ug/cc level. In one embodiment of the present invention, user 13 will not be allowed to administer the bolus drug infusion and exceed the threshold. Thresholds, at which point user 13 may no longer deliver a bolus drug infusion, may be established at any suitable point, where thresholds may vary depending on the age, sex, height, weight, or other physical parameter of patient 18. For example, user 13 may be prevented from administering drugs to a 23 year-old 200 lb. male above a threshold of 20 ug/cc, however user 13 may be prevented from administering drugs to a 70 year-old 120 lb. female above a threshold of 18 ug/cc.

The present invention comprises a plurality of bolus delivery interfaces having a plurality of text boxes and/or buttons, where the bolus delivery interfaces may be used with any suitable user interface 12. A further embodiment of the present invention comprises bolus delivery interfaces where user 13 may input the desired bolus level to be administered. For example, user 13 initiate a bolus delivery interface that requests the amount of drug to be delivered in a bolus infusion. User 13 may signal the preferred bolus drug amount via a touch screen, keypad, voice recognition system, other by other suitable input means, where user 13 may then be prompted to confirm the drug infusion level.

First key 61 and/or second key 62 of third bolus delivery interface 60 may be touch screen buttons that are part of a touch screen display, buttons that are responsive to audio commands, soft buttons, hard buttons, or any other suitable means of inputting a command into sedation and analgesia system 22. In one embodiment of the present invention first key 61 comprises signaling an affirmative response from user 13 to the query presented in third text box 63 and second key 62 comprises signaling a negative response from user 13 to the query presented in third text box 63. First key 61 and/or second key 62 may have textual indicators of their function such as, for example, “Yes” written on first key 61, or iconic indicators of their function such as, for example, an “X” written on second key 62 indicating a negative response. Third text box 63, first key 61, and second key 62 may be positioned at any suitable location on third bolus delivery interface 60 and/or user interface 12.

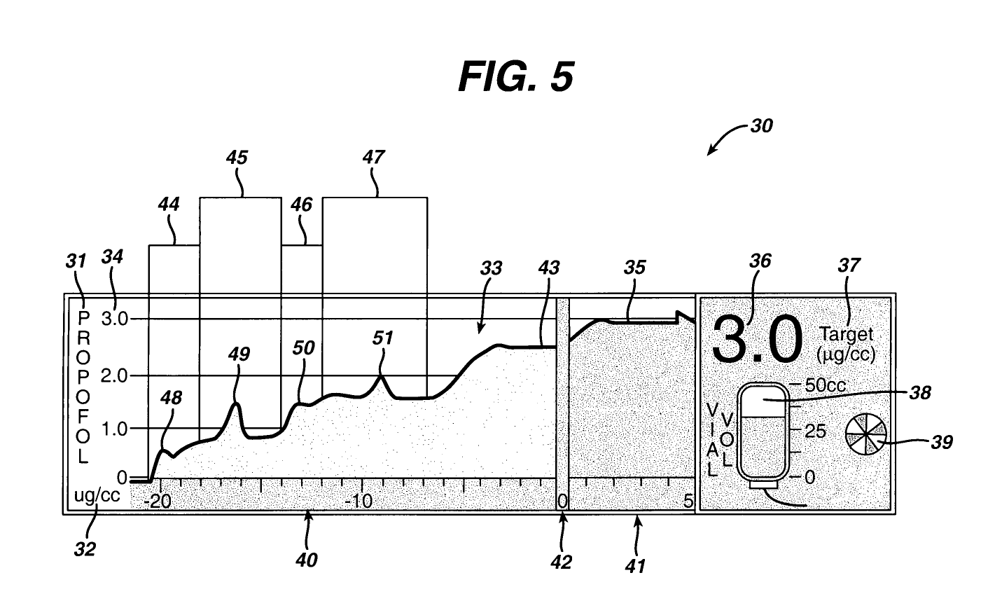

FIG. 5 illustrates one embodiment of drug delivery display 30, where drug delivery display 30 may be integrated with user interface 12 or independent of user interface 12. Drug delivery display 30 may be a incorporated into user interface 12 as a touch screen display, where user 13 may touch icons of drug delivery display 30, or drug delivery display 30 may incorporated into any other visual interface where user 13 inputs data via hard buttons, soft buttons, audio commands, or by any other suitable input means. Embodiment of drug delivery display 30 are illustrated by example only, and do not limit the scope of the present invention.

In one embodiment of the present invention, drug delivery display 30 comprises historical drug data 33, current drug data 43, and anticipated drug data 35. Historical drug data 33 may be presented in the form of a graph, where user 13 may view the changes in drug infusion levels over the course of the procedure or over a sampled portion of the procedure. For example, historical drug data 33 may be programmed to display information from the past 20 minutes of a medical procedure, the past 10 minutes of a medical procedure, or any other desirable period to provide user 13 with a clear picture of the drug infusion regimen administered to patient 18. Data may further be presented in basic numeric characters, in a graph with numeric characters displayed at critical points, or by any suitable means of data display. Current drug data 43 may be a numeric indicator of the present estimated target site concentration of a drug administered to patient 18. Current drug data 43 comprises illustrating the word “CURRENT” to indicate to user 13 the meaning of numeric data presented in current drug data 43. Anticipated drug data 35, in one embodiment of the present invention, comprises programming associated with controller 14, where controller 14 estimates the future effect site concentration of a drug based on the infusion rate established by user 13 and the physical parameters of patient 18. Anticipated drug data 35 may be calculated for any suitable period of time such as, for example, five minutes.

Drug delivery display 30 may further comprise historical timeline 40, current bar 42, and anticipated timeline 41. Historical timeline 40 may be a linear timeline with hash marks at any suitable time measure such as, for example, every minute, where any suitable time measure may be further called out by a numeric indicator such as, for example, a hash mark indicating a period twenty minutes before a current point in the procedure may have “−20” or “−20 min” illustrated above the hash mark. Current bar 42, in one embodiment of the present invention, is a visual bar, where the bar separates historical drug data 33 from anticipated drug data 35. Current bar 42 may be designed in any suitable fashion, however it is preferable to provide a current bar 42 that is easily distinguishable from historical drug data 33 and anticipated drug data 35, where, for example, current bar 42 is a unique color. Current bar 42 may be labeled with “0”, “Current”, or any other suitable indicator capable of informing user 13 that current bar 42 illustrates the present effect site concentration of a drug administered to patient 18. Anticipated timeline 41 may indicate any suitable time period for which controller 14 is programmed to estimate the future effect site concentration of a drug administered to patient 18. Hash marks indicating estimated patient events at a particular time are further consistent with the present invention, where hash marks may be present at any suitable period or periods such as, for example, every minute. Historical timeline 40, current bar 42, and/or anticipated timeline 41 may be positioned at any suitable location on drug delivery display 30 and/or user interface 12.

Drug delivery display 30 may further comprise drug level axis 34, drug label 31, and drug unit 32. Drug level axis 34 is, in one embodiment of the present invention, established as a measure of the effect site concentration of a drug administered to patient 18, where drug unit 32 may display the units such as, for example ug/cc, in which the drug data is being presented. Drug level axis 34 may be designed in any suitable fashion with any suitable increments of drug infusion such as, for example, where every 1 ug/cc increment is called out by a numeric indicator and every 0.5 ug/cc increment is called out by a hash mark. Display 30 may further comprise graph lines associated with drug level axis 34, where graph lines corresponding to effect site concentrations may pass through historical drug data 33 and/or anticipated drug data 35. Drug label 31 may indicate to user 13 the composition of the drug, the generic name of the drug, and/or the brand name of the drug incorporated into sedation and analgesia system 22 for administration to patient 18. In one embodiment of the present invention, sedation and analgesia system 22 is designed to read, for example, a bar code label on a drug to be used in a sedation and analgesia procedure, where the bar code represents drug data that is displayed on drug delivery display 30.

In one embodiment of the present invention, drug delivery display 30 further comprises numeric target infusion level 36, text box 37, vial volume 38, and delivery icon 39. Target infusion level 36 may be a numeric indicator of the target effect site concentration of an administered drug desirable by user 13, where text box 37 displays the function of target infusion level 36 and/or the units in which target level 36 is calculated. Vial volume 38 may be an iconic, numerical, and/or textual indicator of the volume remaining a drug vial, syringe, or other drug delivery device incorporated into sedation and analgesia system 22. Vial volume 38 may be shaped in the form of, for example, a drug vial, where a visual display representing liquid in the vial is measure by hash marks associated with vial volume. For example, a 50 cc vial of propofol may be incorporated into sedation and analgesia system 22, where hash marks associated with vial volume 38 may have numerical indicators representing the volume of drug remaining in the vial. As the vial empties, one color representing drug remaining will line up with the appropriate hash mark for drug volume remaining in the vial, whereas a second color, preferably distinguishable from the color indicating volume, will indicate the amount of drug dispensed. Delivery icon 39 may be a visual display that flashes, revolves, or in any other suitable way indicates that the drug delivery mechanism is operating properly.

In particular embodiments of the present invention, an example of which is depicted in FIG. 5, bolus drug infusions, such as 48, 49, 50, and 51, are delivered at any point during a surgical procedure, where the bolus drug infusions may be represented in historical data display 33. Historical data display 33 and/or anticipated data display 35 may be segmented into various drug delivery modes such as, for example, initial infusion mode 44, first level delivery mode 45, step up mode 46, and second level delivery mode 47. At the beginning of a medical procedure, in accordance with initial infusion mode 44, user 13 may input a target effect site concentration of a drug such as, for example, propofol, where sedation and analgesia system 22 may proceed to deliver a slow ramp up drug increase to reach the desired level, or a higher infusion rate followed by a slower infusion rate to reach the desired effect site concentration more quickly. At any point during initial infusion mode 44, user 13 may administer first bolus drug infusion 48, where first bolus drug infusion may be the amount of drug needed to raise the patient's effect site concentration 0.5 ug/cc. If starting from an effect site concentration of 0 ug/cc user 13 may command first bolus drug infusion 48 to increase the effect site concentration of patient 18 to 0.5 ug/cc. Controller 14 will determine, based on a pharmokinetic model of the drug to be administered and the physical parameters of patient 18, the volume of drug needed to increase the effect site concentration to 0.5 ug/cc. The proper volume of drug may then be delivered by sedation and analgesia system 22, where the infusion will immediately drop to the pre-programmed target infusion rate following the delivery of first bolus drug infusion 45. Providing bolus drug deliveries in such a manner decreases the probability that user 13 will not deactivate an increased volume of drug infusion, thereby decreasing the probability of patient 18 overdosing. Measuring bolus drug infusions in the form of effect site concentrations based on a patient's physical parameters provides a closer estimation of the actual volume of drug needed to achieve a particular sedation or anesthetic effect, thereby reducing the probability of overmedication and under-medication.

A bolus drug infusion may further be given when patient 18 has reached the target effect site concentration, as illustrated by second bolus drug infusion 49 being administered during first level delivery mode 45. This may be performed when user 13 feels patient 18 may need a brief increase in drug level to maintain the comfort, sedation, or anesthesia of patient 18 during an especially painful episode, yet the drug level need not be maintained beyond that brief period. Second bolus drug infusion 49 further illustrates how a bolus drug infusion may be given at any suitable level such as, for example, a bolus infusion substantial enough to create an effect site concentration increase of lug/cc. In one embodiment of the present invention, this is accomplished by confirming two bolus drug infusions, where each bolus drug infusion corresponds to an effect site concentration drug increase of 0.5 ug/cc.

Providing user 13 with the ability to deliver a bolus drug infusion at any time during a medical procedure allows user 13 to administer a drug increase for extremely painful, yet brief, episodes that may occur during some medical procedure such as, for example, cardiac cardioversions. Providing a bolus infusion provides a brief increase in a patient's effect site concentration that quickly begins to drop once the bolus infusion target has been reached. Delivering drugs in such a manner decreases the possibility that an increased drug level that is only needed for a brief period is inadvertently left on. Providing user 13 with the bolus infusion functionality incorporated into a sedation and analgesia system may increase patient comfort and/or maintain sedation or anesthesia during brief periods of extreme pain or discomfort. The bolus infusion functionality further protects patient safety, where the target effect site concentration of a patient will quickly drop following the peak of the bolus infusion, thereby protecting the patient from overdose.

In accordance with the present invention, drug infusions of any magnitude within pre-programmed limits may be administered at any time during a medical procedure as illustrated by third bolus drug delivery 50 being administered during step up mode 46 and fourth bolus drug delivery 50 being administered during second level delivery mode 47. The infusion of third bolus drug delivery 50 represents one example of how user 13 may initiate a bolus infusion while sedation and analgesia system 22 is slowly ramping up towards a newly entered target effect site concentration level. The infusion of fourth bolus drug delivery 51 represents one example of how user 13 may initiate a bolus infusion while sedation and analgesia system 22 is maintaining a particular effect site concentration.

Bolus drug infusions, such as 48, 49, 50, and 51, are illustrated in historical data display 33, where historical data display 33 is illustrated as a graphical display, by example only. Bolus drug infusions may also be illustrated in numerical form, audio form, or by any other suitable display means, and may be displayed in any suitable location on drug delivery display 30 and/or user interface 12.

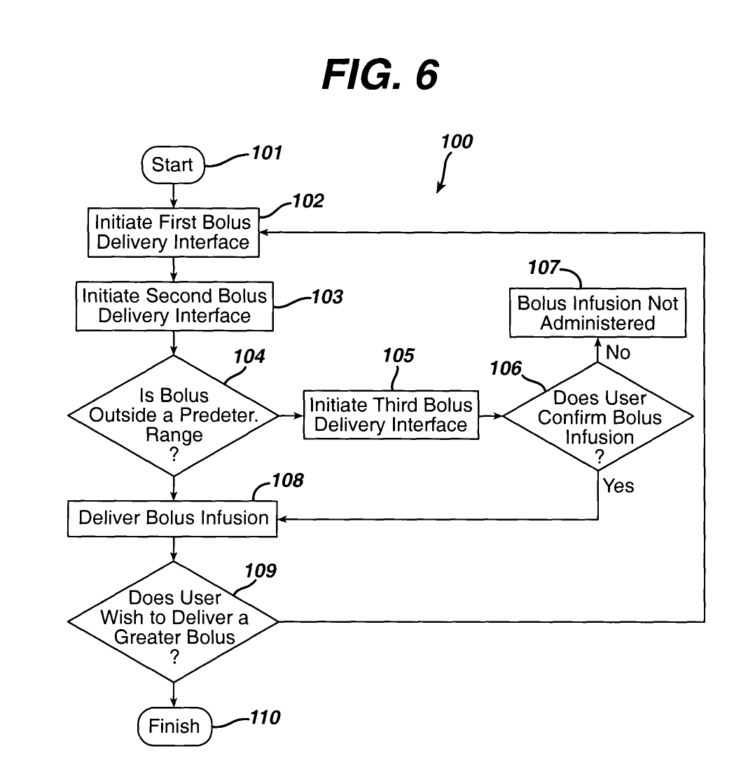

FIG. 6 illustrates one embodiment of method for delivering a bolus drug infusion in cooperation with a sedation and analgesia system, herein referred to as method 100. Step 101 comprises starting sedation and analgesia system 22, attaching patient interface 17 to patient 18, and other procedures necessary to enable the delivery of a bolus drug infusion. Following step 101, method 100 may proceed to step 102, where step 102 comprises user 13 prompting first drug delivery interface 20. First drug delivery interface 20 may be prompted by user 13 depressing a hard button, soft button, touch screen icon, or other suitable means of initiating first drug delivery interface 20. Further, the present invention may provide first drug delivery interface 20 in user interface 12 at all times. User 13 may then select first key 21 to command a bolus drug infusion or second key 23 to cancel first drug delivery interface 20. If user 13 cancels first drug delivery interface 20, method 100 may not proceed. If user 13 commands a bolus drug infusion, method 100 may proceed to step 103.

In one embodiment of the present invention, step 103 comprises prompting second drug delivery interface 25, where user 13 may be required to confirm that they desire a bolus infusion to be delivered to patient 18. User 13 may cancel the infusion command by initiating second key 27, or may confirm the command to deliver a bolus infusion by initiating first key 26. If user 13 cancels the initial bolus infusion request, method 100 will end and may be restarted. If user 13 confirms the initial bolus infusion request, method 100 may proceed to query 104.

In one embodiment of the present invention, query 104 comprises ascertaining whether the bolus drug infusion confirmed by user 13 is outside the pre-programmed range as stored in controller 14. If controller 14 determines that the request is outside the pre-programmed range, method 100 may proceed to step 105.

Step 105 comprises prompting third drug delivery interface 60, where user 13 will be informed that the bolus infusion is outside the range of normally accepted safe drug levels based on the physical parameters of patient 18. In one embodiment of the present invention user 13 may not be allowed to administer a bolus infusion beyond a pre-determined level such as, for example, 20 ug/cc. In a further embodiment of the present invention, user 13 will be required to confirm their decision to deliver an infusion beyond the scope of the pre-programmed safe drug levels as shown by step 106. The bolus infusion outside the pre-programmed safe range may, in one embodiment of the present invention, be canceled by initiating second key 62 of third drug delivery interface 60, or confirmed by initiating first key 61 of third drug delivery interface 60. If user 13 cancels the bolus infusion, method 100 may proceed to step 107, where step 107 comprises not administering the bolus infusion. If user 13 confirms the bolus infusion, method 100 may proceed to step 108. Method 100 may also proceed to step 108 if the bolus infusion associated with query 104 is not outside the pre-determined safe range for drug delivery.

Step 108, in one embodiment of the present invention, comprises drug delivery 19 of sedation and analgesia system 22 delivering a bolus drug infusion to patient 18 based on parameters stored and computed by controller 14. The present invention comprises the infusion of any suitable drug such as, for example, propofol. The present invention further comprises programming associated with controller 14, where controller 14 is programmed to deliver the appropriate drug dosage to achieve a desired effect site concentration of a drug. Following a bolus infusion, method 100 may proceed to step 109.

Step 109, in one embodiment of the present invention, comprises querying user 13 whether they wish to deliver a bolus infusion of greater magnitude. In one embodiment of the present invention, user 13 may input bolus infusions of a consistent magnitude, where user 13 must request multiple infusions to achieve a single bolus infusion of greater magnitude. If user 13 wishes to increase the magnitude of the bolus infusion, method 100 may proceed to step 102, where user 13 will again have to request and confirm a greater bolus infusion. The present invention further comprises querying user 13 whether they would like to initiate a bolus infusion of greater magnitude before step 108, where a single bolus infusion may be made after user 13 has selected the proper bolus infusion level. If user 13 does not wish to increase the magnitude of the bolus infusion, method 100 may proceed to step 110.

Step 110, in one embodiment of the present invention, comprises minimizing or eliminating the drug delivery interfaces from drug delivery display 30 and/or user interface 12. Step 110 further comprises not administering a bolus infusion and/or deactivating sedation and analgesia system 22.

While the present invention has been illustrated by description of several embodiments, it is not the intention of the applicant to restrict or limit the spirit and scope of the appended claims to such detail. Numerous variations, changes, and substitutions will occur to those skilled in the art without departing from the scope of the invention. Moreover, the structure of each element associated with the present invention can be alternatively described as a means for providing the function performed by the element. Accordingly, it is intended that the invention be limited only by the spirit and scope of the appended claims.

What is claimed is:

1. A drug delivery system for a sedation and analgesia system, said drug delivery system including a user interface comprising:
a. a first bolus delivery user interface;
b. a second bolus delivery user interface wherein said second bolus delivery user interface appears after said first bolus delivery user interface to confirm a bolus initiated in said first bolus delivery user interface; and
c. a drug delivery display wherein results are communicated to a user.

2. The drug delivery system of claim 1 further comprising a third bolus delivery user interface wherein said third bolus delivery user interface appears after said second bolus delivery user interface to confirm said bolus if the amount of said bolus is outside a predetermined range; and

3. The drug delivery system of claim 1 wherein said first bolus delivery user interface further comprises of a text box, a yes button, and a cancel button.

4. The drug delivery system of claim 1 wherein said second bolus delivery user interface further comprises of a text box, a yes button, and a cancel button.

5. The drug delivery system of claim 1 wherein said third bolus delivery user interface further comprises of a text box, a yes button, and a cancel button.

6. The drug delivery system of claim 1 wherein said drug delivery display can be used to graphically show bolus delivery.

7. A drug delivery system for a sedation and analgesia system, said drug delivery system including a user interface comprising:
a. a first bolus delivery user interface;
b. a second bolus delivery user interface wherein said second bolus delivery user interface appears after said first bolus delivery user interface to confirm a bolus initiated in said first bolus delivery user interface; and
c. a third bolus delivery user interface wherein said third bolus delivery user interface appears after said second bolus interface to confirm said bolus if the amount of said bolus is outside a predetermined range; and
d. a drug delivery display wherein results are communicated to a user.

8. The drug delivery system of claim 7 wherein said first bolus delivery user interface further comprises of a text box, a yes button, and a cancel button.

9. The drug delivery system of claim 7 wherein said second bolus delivery user interface further comprises of a text box, a yes button, and a cancel button.

10. The drug delivery system of claim 7 wherein said third bolus delivery user interface further comprises of a text box, a yes button, and a cancel button.

11. The drug delivery system of claim 7 wherein said drug delivery display can be used to graphically show bolus delivery.

12. A method of starting a drug delivery system for a sedation and analgesia system which comprises:
a. initiating a first bolus delivery user interface;
b. initiating a second bolus delivery user interface;
c. determining if a bolus is outside a predetermined range programmed by a user; and
d. delivering a bolus infusion.

13. A method of method of starting a drug delivery system for a sedation and analgesia system recited in claim 12 wherein determining if a bolus is outside a predetermined range programmed by a user further includes initiating a third bolus delivery user interface.

14. A method of method of starting a drug delivery system for a sedation and analgesia system recited in claim 13 wherein initiating said third bolus delivery user interface enables said user to cancel said bolus or confirm said bolus.

15. A method of method of starting a drug delivery system for a sedation and analgesia system recited in claim 12 wherein delivering a bolus further includes prompting a user to deliver a greater bolus.

16. A method of method of starting a drug delivery system for a sedation and analgesia system recited in claim 15 wherein prompting a user to deliver a greater bolus further includes returning to said first bolus delivery user interface.
